(19) United States
(12) Patent Application Publication
(10) Pub. No.: US 2004/0220464 A1
(43) Pub. Date: Nov. 4, 2004

(54) Method and apparatus for carrying out a televisit

(75) Inventors: Ralf Benninger, Oberkochen (DE); Michael Haisch, Aalen (DE)

Correspondence Address: CASELLA & HESPOS, 274 MADISON AVENUE, NEW YORK, NY, 10016

(73) Assignee: Carl-Zeiss-Stiftung trading as Carl Zeiss, Heidenheim (DE)

(21) Appl. No.: 10/691,356

(22) Filed: Oct. 22, 2003

(30) Foreign Application Priority Data
Oct. 26, 2002 (DE) 102 50 954.9

(51) Int. Cl. A61B 6/00

(52) U.S. Cl. 600/407; 600/476; 600/473; 128/904

(57) ABSTRACT

The invention concerns a method of carrying out a televisit which comprises the steps: acquiring the data of a patient, which are relevant for the televisit, recording an image of at least one body zone of the patient, which is relevant for the televisit, and transmitting the data and the image to a medical institution. The method according to the invention is distinguished in that a 3D-image is recorded as the image of the body zone of the patient, which is relevant for the televisit.

In addition there is provided a televisit apparatus (1; 100) for carrying out a televisit comprising an acquisition unit (5; 103) for acquiring data of a patient, a transmission unit (9) for sending the data, and a camera (7; 107) for recording an image of at least one body zone of the patient. The apparatus (1; 100) according to the invention is distinguished in that the camera (7; 107) is a 3D-camera.

### METHOD AND APPARATUS FOR CARRYING OUT A TELEVISIT

The present invention concerns a method and an apparatus for carrying out a televisit. In addition the invention concerns a 3D-camera, in particular for use when carrying out a televisit.

The hospital scene in Germany is going to change because of the steadily rising costs involved in the health system. The density of the institutions involved will become less by virtue of lack of profitability or the situation will involve increased specialisation in respect of the individual institutions. From the point of view of the patients that means longer travel distances for operative intervention and difficulties in regard to the people associated with a patient, in terms of being able to visit the patient after an operation and look after the patient. In order to compensate for the reduction in density of the institutions, there is an interest in minimising the length of the stay of a patient in the nursing ward of a hospital before and after the operation.

In addition it is also in the interest of many patients, particularly in the case of children and older people, to return to the accustomed everyday surroundings as quickly as possible, especially as their return additionally has a positively supporting action in terms of the healing process.

The question of aftercare arises for the doctors conducting the treatment and also for the patient, after early discharge. On the one hand the clinic will be too far away for the patient to make a daily visit by ambulance, while on the other hand a frequent house visit on the part of a doctor with his own practice for aftercare purposes would increase the health costs involved.

One possibility of aftercare after early discharge from a hospital is afforded by telemedicine. The term telemedicine is used to denote the application of telecommunication means and information technology in medicine. That includes for example the digital transmission of findings, electronic patient card and records, patient monitoring, digital archiving and so forth. Areas of use of telemedicine are inter alia tele-surgery, -psychiatry, -ophthalmology, -radiology, -pathology, and -traumatology, but also teleconsulting, televisit and homecare.

With the reduction in the period of time for which a patient stays in the nursing ward of a hospital, telemedicine and in particular a televisit will gain in significance as a means for aftercare after an operation. A televisit makes it possible to implement aftercare in the home, without a house visit on the part of the doctor conducting the treatment or a visit on the part of the patient by ambulance being necessary.

Methods and apparatuses for carrying out televisits are known for example from EP 1 062 907, FR 2 760 962, GB 2 288 511, U.S. Pat. No. 5,441,047, U.S. Pat. No. 5,544,649, U.S. Pat. No. 5,961,446 and JP 03198832. Those systems generally offer the option of audiovisual contact between the patient and the medical personnel. The medical personnel can obtain an image of the state of health of the patient by asking the patient. Visual contact with the patient is helpful in that respect. Visual contact can also be used by the medical personnel to obtain an optical impression of zones of the body which are to be investigated.

In comparison with that state of the art the object of the invention is to provide an improved method and an improved apparatus for carrying out a televisit. A further object of the present invention is to provide a camera with which, in the event of a televisit, the options in terms of examining a patient can be enlarged.

The first object is attained by a method of carrying out a televisit according to claim 1 and by an apparatus for carrying out a televisit according to claim 26. The second object is attained by a 3D-camera according to claim 18.

The method of carrying out a televisit includes the following steps: acquiring the data of a patient, which are relevant for the televisit, recording an image of at least one body zone of the patient, which is relevant for the televisit, and transmitting the data and the image to a medical institution. In that respect the medical institution to be considered is any kind of institution in which the transmitted data are kept ready for expert examination by medically trained personnel. The method according to the invention is characterised in that a three-dimensional image (3D-image) is recorded as an image of the body zone of the patient, which is relevant for the televisit.

Recording and transmitting a three-dimensional image enlarges the diagnostic options on the part of the medical personnel carrying out the investigations, for example the doctor performing the treatment. It enables the doctor in particular to obtain a plastic, that is to say three-dimensional impression of the body region of the patient, which is to be investigated. The plastic impression is of particular significance in terms of assessing healing wounds or swellings.

In order to be able to associate the transmitted data with a patient, both personal and also medical data are acquired, as relevant data in respect of the patient.

If the data are acquired as answers to instructions and/or questions presented to the patient, a specific and targeted investigation can be carried out on the basis of those questions or instructions. In particular the questions or instructions can be matched to the present condition of the patient, before or during the televisit. It is therefore advantageous if the questions and/or instructions are respectively transmitted at the beginning of or possibly during the televisit to a televisit device which is disposed at the patient's location. Alternatively the questions or instructions can also be stored in the televisit device. In that way the transmission duration of a televisit can be reduced. The saving on transmission time however is at the expense of flexibility in terms of updating the questions if there is no possibility of transmitting further questions or instructions as the doctor conducting the treatment, when picking out the questions or instructions, is then limited to the catalogue stored in the televisit device.

Transmission of the data and/or questions or instructions to the medical institution or from the medical institution can be effected for example by telephone line, by cellular radio or by the Internet. In order to prevent the data and/or the questions or instructions from passing into the wrong hands, they can be transmitted in encrypted form.

If the transmitted data and/or the questions or instructions are stored in the medical institution, that permits the doctor carrying out the treatment (or other medical personnel) to evaluate the transmitted data when he finds time to do this in his daily routine. In addition, particularly if storage takes place over prolonged periods of time, such storage affords the possibility of documenting the progress in the illness or healing procedure, which is advantageous in terms of assessing the progress involved and makes it possible to carry out a check on the treatment in the case of claims for compensation. In order to prevent the stored data in the medical institution from being called up by unauthorised persons, identification of the person calling up the data can be requested in the call-up procedure before the data is released.

A series of symptoms leads to wounds which heal only very slowly. For example in the case of patients who were bedridden for a long period of time, such symptoms include skin locations which involve bedsores (decubitus) but also wounds and sores in the case of diabetic patients. In order to monitor the healing process the wound must be measured at regular intervals for example in the context of the method according to the invention for carrying out a televisit.

In an advantageous embodiment of the invention therefore measurement of the body zone of the patient, which is relevant for the televisit, is effected on the basis of the 3D-image, for example by means of a photogrammetric evaluation. Measurement is particularly appropriate for evaluating the progress in terms of healing of wounds. To support photogrammetric evaluation, during recording of the 3D-image a pattern can be projected onto the body zone of the patient, which is relevant for the televisit. Photogrammetric evaluation can also be facilitated if the 3D-image is recorded in a wavelength range other than the visible range. However the visible image is also important for assessing for example wound healing processes. In another advantageous embodiment therefore both a two-dimensional image (2D-image) or a 3D-image in the visible wavelength range is recorded and also a 3D-image is recorded in a wavelength range other than the visible range. If the 3D-image is recorded in a wavelength range which is not a visible range, it is advantageous for the pattern also to be projected in the non-visible wavelength range.

In accordance with the invention there is also provided a 3D-camera, in particular a camera for use when carrying out a televisit, comprising an objective and a camera chip, which is distinguished in that the objective includes two recording devices for recording partial images, in particular stereographic partial images of the original of which the image is to be formed, for example a region of the body of the patient, from two different recording directions, wherein a respective partial image is produced for each direction. Recording devices in accordance with the invention can be all devices which are suitable for producing an image of the object to be recorded by means of optical elements, for example lenses, prisms and so forth, on a film plane or a camera chip. The objective is such that both partial images are imaged in mutually juxtaposed relationship or in succession in respect of time on the camera chip. In that respect the reference to in mutually juxtaposed relationship can mean in vertically mutually juxtaposed relationship, in horizontally mutually juxtaposed relationship, or in some other adjacent arrangement.

Conventionally, 3D-recordings of wounds are produced by the wound either being recorded with a single camera in succession from two directions, as is described for example in U.S. Pat. No. 5,976,979, or the wound is recorded by means of two cameras simultaneously from two different directions, as described for example DE 100 21 431. In both cases a specific recording is produced for each of the two partial images. In contrast, the 3D-camera according to the invention provides that both partial images are produced in a single recording and integrated on the chip to form a single overall image. The advantage over the state of the art is that, by virtue of the 3D-image being recorded in a single recording, the 3D-image is not adversely affected by a movement of the part of the body being photographed between two successive recordings and also no adjustment of two separate cameras relative to each other is required. The image can then be fed to a computer where it is broken down again into its partial images and photogrammetric measurement of the wound is effected. Fixing of the body region to be recorded is eliminated by virtue of simultaneous recording of the stereoscopic partial images.

Instead of only two recording devices the 3D-camera according to the invention may also have more than two recording devices for recording partial images of the body region or original of which the image is to be produced from more than two different recording directions, with a respective partial image being produced for each direction. The recording devices can then be of such a configuration that all partial images are produced in mutually juxtaposed relationship on the camera chip.

Each recording device may be provided with its own optical system, that is to say for example its own front and its own rear lenses. Alternatively, instead of completely separate optical systems, it is also possible to use in part a large optical system, that is to say each recording device has for example its own front lens, whereas the rear lens is a common large lens for all the recording devices.

In order to support photogrammetric evaluation of the 3D-recording, it is advantageous if, during the recording procedure, a pattern is projected onto the original or the region of the body which is to be recorded. For projecting such a pattern the 3D-camera may include a projection system. A symmetrical recording of the original is achieved if the recording devices are arranged in mirror image symmetry relative to each other and the projection system is arranged in the mirror plane between the recording devices. In that case both partial images are recorded at the same angle relative to the projected pattern.

In addition, for photogrammetric evaluation of a 3D-image, it may be advantageous if the image has been recorded in a wavelength range other than the visible range. Therefore the projection system can be designed for projection in a wavelength range outside the wavelength range of visible light.

In order to permit simultaneous recording in the visible and a non-visible wavelength range, at least one recording device of the 3D-camera can include a splitter for separating the partial image produced by the recording device into a visible partial image in the visible wavelength range and an invisible partial image outside the wavelength range of visible light. The beam path in the splitter is then such that the invisible partial image has the image thereof formed on the camera chip beside the visible partial images.

In accordance with the invention in addition there is provided a televisit apparatus for carrying out a televisit, comprising an acquisition unit for acquiring data of a patient, a transmission unit for transmitting the data and a camera for recording an image of at least one body zone of the patient. In that respect the term acquisition unit is used to denote all units, with which data or details of a patient can be acquired. Therefore, both keyboards or microphones and also measuring devices such as for example thermometers, blood pressure measuring devices, diabetes measuring devices etc. can be involved as the acquisition units. The apparatus according to the invention is distinguished in that the camera is a 3D-camera.

For carrying out a televisit, it is advantageous if there is the option on the part of the patient to answer questions or carry out instructions. For that purpose the televisit apparatus can include an output unit, for example a monitor or a loudspeaker, for outputting questions and/or instructions to the patient. The acquisition unit and the output unit can also be embodied in the form of a single device such as for example a touch-sensitive screen (touchscreen).

In addition the televisit apparatus may also have at least one connection for the connection of at least one further acquisition unit or output unit. In that way it is possible for at least one measuring device to be connected at the same time in addition for example to a keyboard or a speech input unit. If the apparatus has a plurality of connections, it is possible to avoid changing the measuring device during the televisit.

The televisit apparatus may include a memory for the storage of questions and/or instructions. In addition the televisit apparatus may include a receiving unit for receiving questions and/or instructions to the patient. In that way it is not only possible for questions and/or instructions stored in the apparatus to be outputted to the patient, but also questions and/or instructions which the doctor carrying out the treatment communicates to the patient on the basis of the current situation prior to or during the visit.

The transmitting and/or receiving unit can be adapted for transmission and/or reception by way of a telephone line.

A televisit apparatus must be ready for use at all times and everywhere. It is therefore particularly advantageous for the transmitting and/or receiving unit to be adapted for transmitting and receiving by way of a cellular network. A cellular connection affords a number of advantages in comparison with a fixed network connection by way of the telephone connection socket. Often, articles of furniture are positioned in front of a telephone connection socket or the plug connection is already in use by another piece of equipment such as for example a telephone or a fax machine. In addition, particularly in older buildings, the telephone connection sockets are mostly disposed only in the hall or in the living room area, which can give rise to problems if the patient is bedridden. Extension cables are not an acceptable solution because of the risk of an accident that they entail. The question of ISDN or analog connection in the case of a fixed network connection can also give rise to complications as the correct modem has to be available. Connecting the televisit apparatus to the cellular network in contrast makes the apparatus independent of location and means that it can be used in a flexible manner. Even if the patient is travelling, it is possible at any time to make a connection with the medical institution which is providing for patient care. It is therefore particularly advantageous if the televisit apparatus not only permits communication by way of a cellular network but is itself in the form of a mobile televisit apparatus, for example in the form of a WebPad, a PDA (Personal Digital Assistant), a mobile telephone or a notebook.

In order to protect the data transmitted by the transmitting unit from unauthorised access by third parties, the televisit apparatus may include an encryption unit for encryption of the data to be transmitted. Correspondingly the televisit apparatus can include a decryption unit for decryption of the received questions and/or instructions. The encryption and decryption units can be embodied both in the form of hardware and also software.

Further features, properties and advantages of the invention will be apparent from the detailed description hereinafter of embodiments with reference to the accompanying drawings in which:

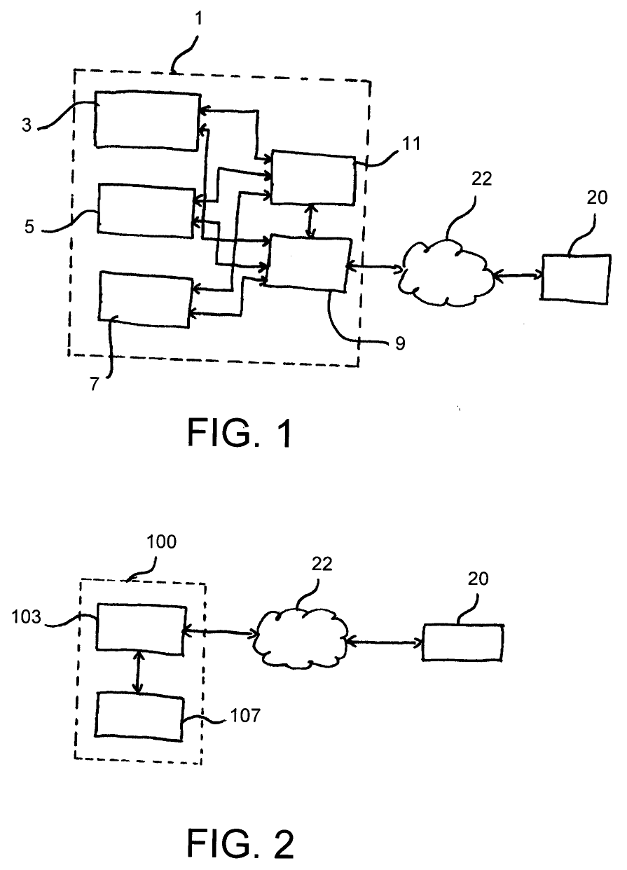

FIG. 1 shows a first embodiment by way of example of the televisit apparatus according to the invention in the form of a block circuit diagram, FIG. 2 shows a second embodiment by way of example of the televisit apparatus according to the invention in the form of a block circuit diagram,

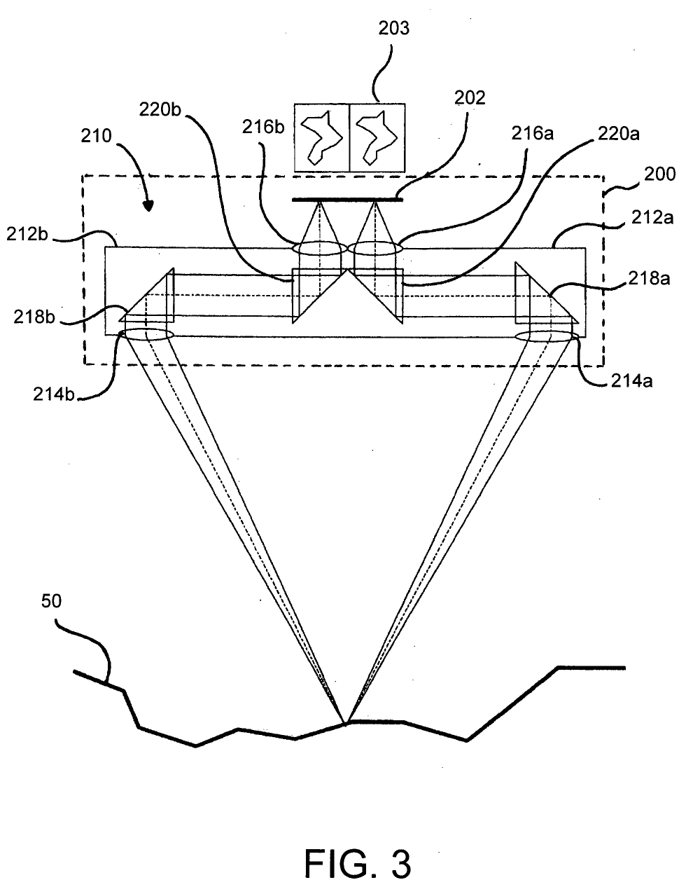

FIG. 3 shows a first embodiment of a 3D-camera,

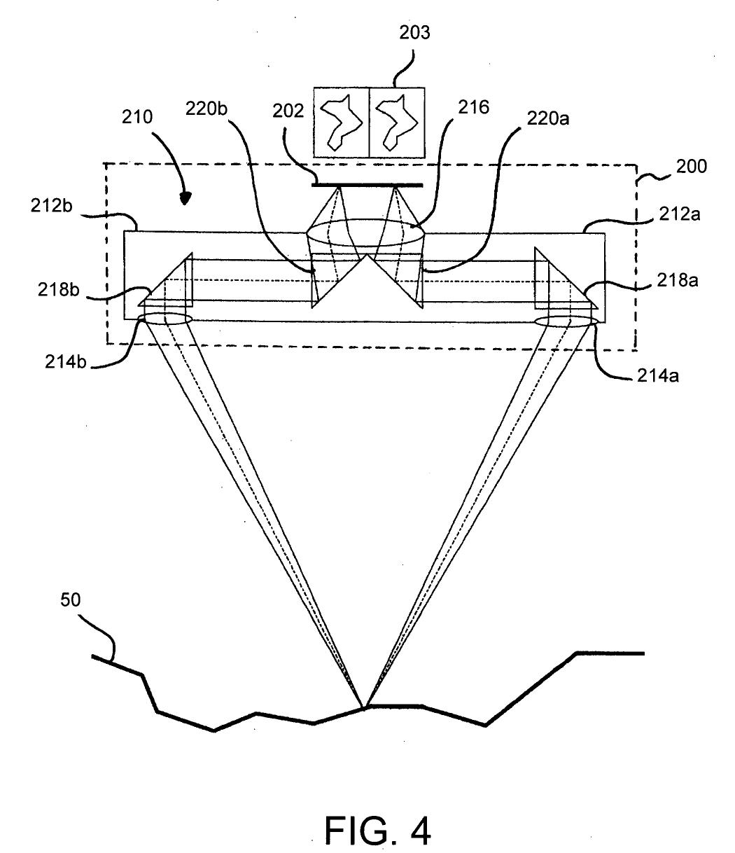

FIG. 4 shows a second embodiment of a 3D-camera,

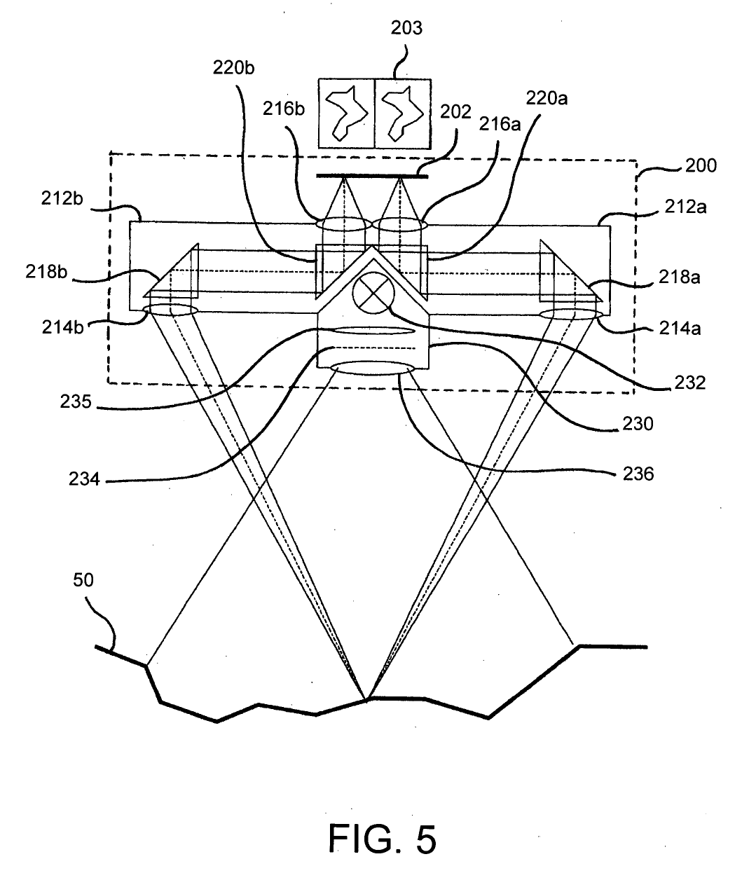

FIG. 5 shows a third embodiment of a 3D-camera, and

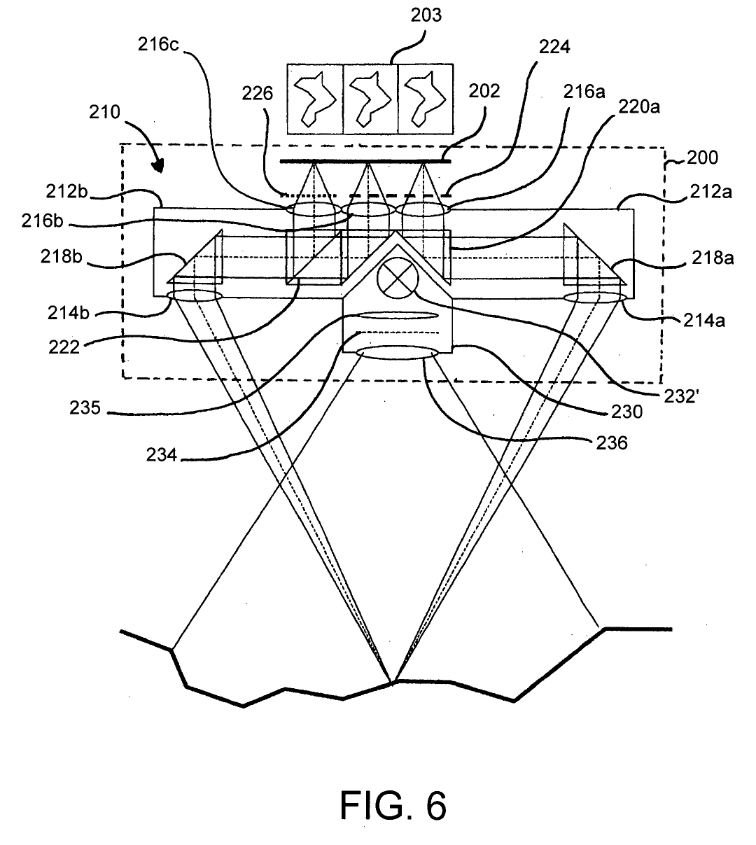

FIG. 6 shows a fourth embodiment of a 3D-camera.

A first embodiment of the televisit apparatus according to the invention will now be described by reference to the block circuit diagram shown in FIG. 1. The televisit apparatus 1 includes a monitor 3 as an output unit, a keyboard 5 as an acquisition unit, a 3D-camera 7 and a transmitter/receiver 9 as a transmitting and receiving unit. The transmitter/receiver 9 is connected to the display screen 3, the keyboard 5 and the 3D-camera 7 and serves on the one hand for transmitting the data acquired by the keyboard 5 and the 3D-camera 7 to a medical institution 20 and for receiving questions and instructions which are transmitted for example by a doctor present in a medical institution 20. Instead of the keyboard 5 or in addition to the keyboard 5 it is also possible to employ other data input devices, for example a touch-sensitive display screen (touchscreen), as the acquisition unit.

In the present embodiment the Internet 22 is selected as the transmission medium. Alternatively transmission can also be effected by way of a direct connection, for example by way of a public telephone dial-up network or a cellular network. In addition the televisit apparatus 1 includes a control unit 11 connected to all other elements of the televisit apparatus 1 by way of control lines, for controlling the processes within the televisit apparatus 1.

The monitor 3 is connected to the transmitting/receiving unit 9 and serves for displaying questions and instructions from the doctor performing the treatment. In addition it can reproduce an image of the doctor. Usually the monitor 3 will also be provided with a loudspeaker so that the doctor can put his questions and give his instructions verbally. The patient gives the answers to the questions for example by means of the keyboard 5 or by another data input device which is present. If a microphone is provided as an optionally additional acquisition unit, the answers can also be transmitted to the doctor verbally. The image of the doctor and the possibility of being in contact with the doctor by way of a loudspeaker and a microphone are intended to help in giving a feeling of personal care in relation to the patient. However a direct connection between the doctor and the patient is not absolutely necessary in the context of a televisit.

The 3D-camera 7 can be in the form of a video camera or a still camera. It serves primarily to record the regions of the body of the patient, which are relevant for a televisit, for example a wound which is in the course of healing. It is preferably a digital 3D-compact camera and can include a zoom optical system and an autofocus function in order to ensure ease of operation. It is particularly advantageous if the shutter time is set automatically and if required a flashlight is activated automatically as that further simplifies operation. The 3D-camera can either be directly integrated into the televisit apparatus 1 but alternatively it can also be connected by cable or wirelessly to the control unit 11 and the transmitter/receiver 9.

The patient or a carer, merely on the basis of the image portion which he can preferably see both on the monitor 3 and also in the viewfinder of the 3D-camera 7, only needs to select the desired image portion and then actuate the shutter. After the 3D-image recorded has been transmitted by the transmitter/receiver 9 to the medical institution 20, the doctor, on the basis of that 3D-image, by means of a 3D-viewing apparatus can obtain a plastic impression of the recorded region of the body, which is important in particular for judging wounds and swellings. It is also possible by means of special programs on the basis of the 3D-image to implement photogrammetric measurement of the recorded region of the body. It is also possible to label the 3D-image by means of an additional program. If the doctor is out or if no 3D-viewing device is available for some other reason, he can also view the image in two-dimensional quality for example on a laptop. After analysis of the 3D-image the image data can be stored in an image archiving system. In addition, there can be hardware and/or software with which the individual 3D-images can be put together to form a film. As the recordings are not always from exactly the same direction, the individual 3D-images are turned by an algorithm in such a way that the surfaces which are imaged therein involve the same spatial orientation. The differing orientations of two successive representations are ascertained with the algorithm and one of the two representations is rotated for example by means of a least square fit until the orientation thereof coincides.

In the time in which no 3D-image of a given region of the body is recorded or if such a recording is not necessary, the 3D-camera 7, particularly if it is in the form of a video camera, can be used to take a portrait of the patient which is transmitted to the doctor. If at the same time a portrait of the doctor is transmitted to the patient, the televisit apparatus 1 is particularly good at affording the impression of a personal dialog between the doctor and the patient. The 3D-functionality of the 3D-camera 7 can be switched off to record the portrait. In that way it is possible to avoid acquiring image information which is irrelevant, in order to keep down the time and expenditure involved in acquiring and sending the portrait.

It is desirable for the televisit apparatus 1 to be also equipped with a memory (not shown) in which the questions or instructions can be stored and in which optionally the answers and/or the data in respect of the patient are stored for despatch if they cannot be sent immediately.

The use of the televisit apparatus 1 will be described hereinafter by way of example with reference to aftercare subsequently to an operation. Depending on the respective state of health or age of the patient, the patient himself or a carer uses the televisit apparatus 1 on a regular basis, for example daily, to produce a three-dimensional recording of the state of his injury, and sends that recording together with a completed questionnaire to the doctor performing his treatment, by way of a secure Internet connection. The questionnaire in principle includes the questions of a daily hospital visit: “how have you slept? Do you have any fever? Do you have any pain, if so, how much (scale on 1 to 10)?” etc. Alternatively the patient can also send the doctor an e-mail in which he sets out his personal state of heath. The 3D-image of the wound and the answers to the questions afford the doctor carrying out the treatment the opportunity to observe the healing process, document it, archive it and optionally take action in good time.

The televisit also affords the hospital doctor with additional advantages. If a patient has assessed his pain or fever with the answer yes and at a very high level (on the basis of a threshold defined by the doctor), then the doctor receives a special notification from his televisit receiving device and he is in a position to make contact with the patient immediately. Otherwise, the doctor has the possibility of evaluating the answers and 3D-images stored in his televisit receiving device, when he finds time to do that in his daily routine. In addition the doctor has the possibility of storing the daily 3D-images in an image archive in order better to evaluate the course of the illness and to be able to document same for any claims for compensation. In addition, if he considers it necessary, he can send the patient data to a colleague for example by way of a secure Internet connection in order to obtain a second opinion.

The patient regularly receives from his doctor an e-mail about the progress in his recovery. He receives for example fresh instructions in regard to wound treatment or the assurance that the healing process is going ahead. The patient and his relatives at any event feel that they are being well cared for and are psychologically strengthened.

It will be appreciated that the televisit can also be used after operations carried out on an outpatient basis. In relation to certain target groups, for example older people, the televisit can also be used preventatively, that is to say as a means for health care or for monitoring an illness which is in a non-critical phase. The doctor performing the treatment is able to observe the state of health on a regular basis, for example daily or weekly, and possibly intervene if he detects a critical stage being reached. Thus in particular when dealing with older people it is possible for a long-term stay in a hospital, which in most cases weakens such people and causes them severe psychological stress, to be entirely avoided under some circumstances.

In the described embodiment the televisit apparatus includes a keyboard as the acquisition unit and a display screen as the output unit. In an alternative configuration however, instead of separate input and output units, the televisit apparatus may also include an integrated input/output unit, for example a touch-sensitive display screen (touchscreen). In addition it may include as many further acquisition units as the control system is capable of controlling. Measuring devices such as for example fever thermometers, diabetes measuring devices, blood pressure measuring devices etc. can be considered as additional acquisition units in that respect. In order not to overburden the patient and in order to avoid making the televisit apparatus unnecessarily complex however it may be appropriate that not all additional acquisition units are connected at the same time. Thus for example a diabetes measuring device is redundant when dealing with a patient who is not a diabetic. It is therefore advantageous if the televisit apparatus is provided with an interface to which additional acquisition units can be connected only when required.

A second embodiment of the televisit apparatus according to the invention is shown in FIG. 2. The acquisition unit, the output unit, the control unit and the transmitting/receiving unit of the televisit apparatus 100 are integrated in a WebPad 103. The function of a combined input and output unit is performed by a touch-sensitive display screen (touchscreen), by means of which it is possible to obtain information about the patient, in respect of his state of health. For that purpose, a clearly understandable input mask can be represented on the touch-sensitive screen. The WebPad 103 must be light, easy to handle and insensitive to shock as it must also be suitable for operation by inexperienced people without the risk of a defect as a result of operator error. It has an interface for the connection of at least one external 3D-camera 107 which is connected to the WebPad 103 for carrying out the televisit. Other acquisition units can be connected to the interface if required. Depending on the respective nature of the interface it is also possible for a plurality of acquisition devices to be connected at the same time, for example the 3D-camera 107 and in addition a diabetes measuring device. The transmitting/receiving unit which is integrated into the WebPad 103 is adapted for transmission and reception by way of a cellular network.

The televisit apparatus can be used in particular irrespective of location if the integrated transmitting-receiving unit is adapted for transmitting and receiving using different cellular standards, for example GSM and ATM. In Germany, two separate cellular network standards based on the GSM standard are in use for the transmission of large amounts of data, namely the HSCSD standard (High Speed Circuit Switched Data, 14.4 Kbit/s with channel bundling) which is operated by D2 and E-Plus, and the GPRS standard (GPRS: General Packet Radio Service, with up to 53.6 Kbit/s) which is operated by D1 and E2. The GPRS standard uses the time slots of the GSM channel, which are not occupied by speech telephone conversations, that is to say it is not possible to guarantee a given speed. The essential advantage of GPRS over HSCSD is that the costs of the connection are not calculated according to the duration thereof but according to the amount of data transmitted. The planned UMTS networks (Universal Mobile Telecommunication System) are intended to gradually replace the existing cellular networks on a worldwide basis. UMTS permits two modes of operation, namely FDD (Frequency Division Duplex) and TDD (Time Division Duplex). In the case of the FDD transmission and reception are effected on different frequencies, wherein the data transfer speed is up to 384 Kbit/s, whereas in the case of TDD transmission and reception are effected on the same frequency and a data rate of up to 2 Mbit/s is possible.

For data protection reasons a particular degree of security must be guaranteed for the transmission of patient data by way of the Internet. The data must be non-readable over the entire transmission path. Therefore the data are sent in encrypted form by the transmitting unit and indeed independently of whether access to the Internet is by way of a telephone line or by means of cellular radio. In a cellular radio device an encryption code can be stored for that purpose on the SIM card so that it is not generally accessible. The encrypted data are sent to a patient server in the medical institution, from which they can be called up by a doctor if he has a suitable private key for identification purposes. The private key is managed by a server which is connected to the patient server.

Various embodiments of the 3D-camera are described hereinafter.

A first embodiment of a 3D-camera which can be used in the televisit apparatus according to the invention is diagrammatically shown in FIG. 3. The Figure shows a wound 50, a 3D-camera 200 and the beams of the two stereoscopic partial images when recording a three-dimensional (stereoscopic) image. In the Figure the wound 50 represents an example of a region of the body which is to be three-dimensionally recorded.

The camera 200 includes a camera chip 202 and an objective 210 which in turn includes a first recording device 212a and a second recording device 212b in a mirror image symmetrical arrangement. The two stereoscopic partial images, as indicated by the illustrated beams, are imaged in mutually juxtaposed relationship on the camera chip 202 by the two recording devices 212a and 212b. A plan view 203 onto the camera chip 202 together with the two partial images is shown in the Figure above the camera chip.

In order to produce the image of the object light indicated by the beams on the camera chip 202, provided in each of the two recording devices 212a, 212b are a front lens 214a, 214b and a rear lens 216a, 216b. Arranged between the front and rear lenses are respective front deflection prisms 218a, 220a and rear deflection prisms 218b, 220b. The terms “front deflection prisms” and “rear deflection prisms” in this respect do not refer to the spatial arrangement of the prisms but to the sequence in which the beams pass therethrough. After passing through the front lens 214a, 214b the rays of the two beams emanating from the wound are parallel. Those parallel rays are deflected by the front deflection prisms 218a, 218b at a right angle in a direction onto the centre of the objective 210 where the rear deflection prisms 220a, 220b are arranged in mutually juxtaposed relationship. The rear deflection prisms 220a, 220b again deflect the beams at a right angle, namely towards the rear lens 216a, 216b. They are then focused in mutually juxtaposed relationship onto the camera chip 216 by the rear lens. The illustrated objective 210 produces a stereoscopic basis for the three-dimensional imaging action, the size thereof depending on the spacing of the two front deflection prisms 214a, 214b from each other.

The objective 210 is of a mirror image symmetrical configuration in the described embodiment of the 3D-camera. That has the advantage that two identical recording devices can be used for forming the objective. It is however also possible for the objective 210 to be of an asymmetrical configuration. Also, it is not absolutely necessary for the deflection prisms to deflect the beams at a right angle. Other deflection angles are also possible, but with the same stereoscopic basis, in relation to the right deflection angle, result in the objective being of a greater structural depth.

A second embodiment of the 3D-camera is shown in FIG. 4. That embodiment differs from the 3D-camera shown in FIG. 3, in that, instead of two separate rear lens, there is a single large rear lens 216 for the beams of the two stereoscopic beam portions. In addition the two rear prisms 220a, 220b are of such a configuration that they do not deflect the rays of the beams at a right angle but through an angle which is somewhat smaller than 90 degrees, in order to take account of the fact that focusing onto the camera chip 202 is effected by means of a common lens. In other respects the embodiment shown in FIG. 4 does not differ from that shown in FIG. 3, and it is therefore not further described herein.

FIG. 5 shows a third embodiment of the 3D-camera. The third embodiment differs from the first embodiment shown in FIG. 3 in that at the centre of the objective 210 is arranged a projection system 230 for projecting a pattern onto the wound. The projection system 230 includes a light source 232, for example a halogen lamp, a grid 234 and a projection lens 236 for projecting an image of the grid onto the wound 50. In order to illuminate the grid 234 uniformly a condenser lens 235 is arranged between the light source 232 and the grid 234. Arranging the projection system 230 at the centre of the objective is not absolutely necessary but advantageous as then both recording devices 212a, 212b record the pattern at the same angle. In other respects the third embodiment does not differ from the first embodiment so that it will not be discussed in further detail herein.

FIG. 6 shows a fourth embodiment of the 3D-camera. In this embodiment the projection system 230 includes a light source 232′ which emits light in an invisible wavelength range, for example in the infrared wavelength range, so that the pattern is projected in that wavelength range onto the wound 50. In one of the two recording devices, in the present embodiment the left-hand recording device 212b, disposed in front of the rear deflection prism 220b is a beam splitter 222 which divides the beam and deflects the one part into a third rear lens 216c. The other part of the beam passes without deflection into the rear prism 220b from which it is finally deflected towards the rear lens 216b. In the other recording device 212a, the beam path is as in the embodiments described hereinbefore.

Arranged between the rear lenses 216a and 216b and the camera chip 202 is a filter 224 which only passes the infrared light so that two infrared partial images are produced on the corresponding regions of the camera chip 202, that is to say a stereoscopic image in the infrared wavelength range is produced. The beam which is branched off in the left-hand recording device in front of the rear deflection prism 220b is imaged by the third rear lens 216c as a third partial image beside the two stereoscopic partial images on the camera chip 202. That beam which produces the third partial image, before impinging on the camera chip, passes through a VIS-filter 226 which filters the invisible wavelengths out of the beam.

The 3D-camera in accordance with the fourth embodiment provides that three partial images are formed in mutually juxtaposed relationship on the camera chip 202, two stereoscopic partial images in an invisible wavelength range and a two-dimensional image in the visible wavelength range. The stereoscopic partial images can be used for photogrammetric evaluation of the wound while the two-dimensional image in the visible wavelength range can be used for visual expert examination of the wound by a doctor.

In the described fourth embodiment, a beam splitter 222 is arranged only in one recording device. However, division of the beam can be related to a loss in intensity in the corresponding stereoscopic partial image, which does not occur in the other stereoscopic partial image. In order to ensure the same level of intensity for both stereoscopic images, a corresponding beam splitter (not shown in the Figure) can also be arranged in the other recording device 212a. The beam which is divided by that beam splitter out of the beam path of the stereoscopic partial image can either be discarded or the image thereof can be formed by means of a further rear lens beside the other three partial images on the camera chip 202. In particular it can first pass through a filter which filters out the invisible wavelengths. That procedure affords a further partial image which, together with the other partial image, in the visible wavelength range, forms a 3D-image. That therefore gives both a 3D-image in the invisible wavelength range and also a 3D-image in the visible wavelength range.

In an alternative configuration of the fourth embodiment the beam splitter can also be designed for wavelength-dependent splitting of the beam, that is to say it deflects for example the light in the invisible wavelength range to a degree of almost 100% and it allows the light in the visible wavelength range to pass without deflection to a degree of almost 100%. A substantial loss in intensity then occurs neither in the visible partial image nor in the invisible partial image. In addition that configuration also affords the possibility of optionally foregoing the use of the filters 224, 226 shown in FIG. 6.

In the described embodiments, all partial images are produced on the camera chip in mutually juxtaposed relationship. Instead of the spatial separation of the partial images however, separation of the partial images in respect of time is also possible. The partial images are then projected individually on succession onto the same region of the camera chip, preferably onto the entire camera chip. Separation of the partial images in respect of time is effected by way of cyclically operating interruption means, for example mechanical shutters or electro-optical switches, or a mirror mechanism which deflects the respective partial image to be recorded in a direction towards the camera chip.

1. A method of carrying out a televisit comprising the steps of:
acquiring the data of a patient, which are relevant for the televisit,
recording an image of at least one body zone of the patient, which is relevant for the televisit, and
transmitting the data and the image to a medical institution, characterised in that
a 3D-image is recorded as the image of the body zone of the patient, which is relevant for the televisit.

2. A method according to claim 1 characterised in that both personal and also medical data are acquired as relevant patient data.

3. A method according to claim 1 characterised in that the data are acquired as reactions to questions and/or instructions presented to the patient.

4. A method according to claim 3 characterised in that the questions and/or instructions are transmitted at the beginning of or during the televisit to a televisit device which is disposed at the location of the patient.

5. A method according to claim 3 characterised in that the questions and/or instructions are stored in a televisit device disposed at the location of the patient.

6. A method according to claim 1 characterised in that the transmitted data are stored in the medical institution.

7. A method according to claim 1 characterised in that the data and/or the questions or instructions are transmitted to the medical institution by telephone line.

8. A method according to claim 1 characterised in that the data and/or the questions or instructions are transmitted by cellular radio.

9. A method according to claim 1 characterised in that the data and/or the questions or instructions are transmitted by way of the Internet.

10. A method according to claim 1 characterised in that the data and/or the questions or instructions are transmitted in encrypted form.

11. A method according to claim 1 characterised in that for calling up the data in the medical institution an identification of the person performing the calling-up procedure is interrogated.

12. A method according to claim 1 characterised in that during recording of the 3D-image a pattern is projected onto the body zone of the patient, which is relevant for the televisit.

13. A method according to claim 12 characterised in that the 3D-image is recorded in a wavelength range other than the visible range.

14. A method according to claim 12 characterised in that the both a 2D-image or a 3D-image is recorded in the visible wavelength range and a 3D-image is recorded in a wavelength range other than the visible wavelength range.

15. A method according to claim 12 characterised in that the pattern is projected in the invisible wavelength range.

16. A method according to claim 1 characterised in that measurement of the body zone of the patient, which is relevant for the televisit, is effected on the basis of the 3D-image.

17. A method according to claim 1 characterised in that a plurality of 3D-recordings are put together to form a film.

18. A 3D-camera, in particular for use in carrying out a televisit, comprising an objective (210) and a camera chip (202), characterised in that the objective (210) includes two recording devices (212a, 212b) for recording partial images of an original (50) of which the image is to be produced, from two different recording directions, wherein a respective partial image is produced for each direction, and the objective (210) is such that both partial images are produced on the camera chip (202) in mutually juxtaposed relationship.

19. A 3D-camera according to claim 18 characterised in that the objective (210) has more than two recording devices (212a, 212b) for recording partial images of an original (50) of which the image is to be produced, from more than two different recording directions, wherein a respective partial image is produced for each direction, and the recording devices (212a, 212b) are so designed that all partial images are produced on the camera chip (202) in mutually juxtaposed relationship.

20. A 3D-camera according to claim 18 characterised in that each recording device (212a, 212b) has its own front and its own rear lens (214a, 214b, 216a, 216b).

21. A 3D-camera according to claim 18 characterised in that each recording device (212a, 212b) includes its own front lens (214a, 214b) and all recording devices (212a, 212b) include a common rear lens (216).

22. A 3D-camera according to claim 18 characterised in that it includes a projection system (230) for the projection of a pattern onto the original (50) of which the image is to be produced.

23. A 3D-camera according to claim 22 characterised in that the recording devices (212a, 212b) are arranged in mirror image symmetry relative to each other and the projection system (230) is arranged in the plane of symmetry of the recording devices (212a, 212b).

24. A 3D-camera according to claim 22 characterised in that the projection system (230) is adapted for projection in a wavelength range outside the wavelength range of visible light.

25. A 3D-camera according to claim 18 characterised in that at least one recording device (212b) includes a splitter (222) for separating the partial image produced by the recording device (212b) into a visible partial image in the visible wavelength range and an invisible partial image outside the wavelength range of visible light and the beam path in the splitter (222) is such that the invisible partial image is produced on the camera chip (202) beside the visible partial images.

26. Apparatus for carrying out a televisit comprising:
an acquisition unit (5; 103) for acquiring data of a patient,
a transmission unit (9) for sending the data, and
a camera (7; 107) for recording an image of at least one body zone of the patient, characterised in that
the camera (7; 107) is a 3D-camera.

27. Apparatus according to claim 26 characterised in that there is provided an output unit (3, 103) for the output of questions and/or instructions to the patient.

28. Apparatus according to claim 27 characterised in that the acquisition unit and the output unit are formed by a touch-sensitive display screen.

29. Apparatus according to claim 28 characterised in that it includes at least one connection for the connection of at least one further acquisition unit or output unit.

30. Apparatus according to claim 29 comprising a blood pressure or diabetes measuring device as a further acquisition unit.

31. Apparatus according to claim 26 characterised in that there is a receiving unit (9) for receiving questions and/or instructions to the patient.

32. Apparatus according to claim 26 characterised in that the transmitting and/or the receiving unit (9) are adapted for transmission and/or reception by way of a telephone line.

33. Apparatus according to claim 26 characterised in that it is in the form of a mobile televisit device.

34. Apparatus according to claim 33 characterised in that the transmitting and/or receiving unit (9) is or are adapted for transmission and/or reception by way of a cellular network.

35. Apparatus according to claim 26 characterised in that there is provided an encryption unit for encryption of the data to be transmitted.

36. Apparatus according to claim 26 characterised in that there is provided a decryption unit for decryption of the received questions and/or instructions.

37. A televisit receiving device for receiving the data sent by an apparatus according to claim 26 characterised in that it includes a module for assembling received 3D-images to form a film.

38. A televisit receiving device according to claim 37 characterised in that it includes a transmitter for transmitting questions and/or instructions.
